(19) United States
(12) Patent Application Publication
(10) Pub. No.: US 2004/0186310 A1
(43) Pub. Date: Sep. 23, 2004

(54) Process for preparation of cyclohexanol derivatives

(76) Inventors: Keun-Sik Kim, Jeollonam-do (KR); Kwang-Il Kim, Jeollonam-do (KR); Sung-Woo Lee, Jeollonam-do (KR); Jin-Soo Park, Jeollonam-do (KR); Ki-Byung Chai, Jeollonam-do (KR)

Correspondence Address: WYETH, PATENT LAW GROUP, 5 GIRALDA FARMS, MADISON, NJ, 07940, US

(21) Appl. No.: 10/481,679

(22) PCT Filed: Jun. 21, 2002
(86) PCT No.: PCT/US02/19753

(30) Foreign Application Priority Data
Jun. 22, 2001 (KR) 10-2001-35889

(51) Int. Cl. C07C 253/30

(52) U.S. Cl. 558/371

(57) ABSTRACT

A process for the preparation of cyclohexanol derivatives of formula (I) by reacting a compound of formula (II) with a compound of formula (III) in the presence of a base catalyst of formula (IV) or (V). In the above formula, R₁-R₉, A, B, X and p have tghe meanings given in the specification.

### BACKGROUND OF THE INVENTION

1. Field of the Invention

The present invention relates to a process for preparation of cyclohexanol derivatives such as 1-[cyano (4-methoxyphenyl)methyl] cyclohexanol.

2. Background of the Related Art

Cyclohexanol derivatives such as 1-[cyano(4-methoxyphenyl)methyl] cyclohexanol are useful intermediates for making compounds like venlafaxine which have anti-depressant effects by inhibiting re-uptake of neurotransmitters, norepinephrine and serotonin. As disclosed in U.S. Pat. No. 4,535,186, cyclohexanol derivatives can be produced by reaction of a cycloalkanone or cycloallkenone with an appropriately substituted (orth-o or para) phenylacetonitrile anion.

The preparation method disclosed in the U.S. Pat. No. '186 patent involves the use of an organometallic base such as n-butyl lithium in order to induce phenylacetonitrile anion in the reaction. The organometallic base is expensive, has to be used in an amount of at least one equivalent of the reactant at a low temperature below −50° C., is characteristically susceptible to water in air with the risk of fire or explosion, and provides a low production yield of less than 50 percent. Therefore, organometallic bases are considered impractical for industrial scale synthesis.

U.S. Pat. No. 5,043,466 discloses a process for preparation of cyclohexanol derivatives which use an organometallic base such as a lithium diisopropylamide as illustrated in the following reaction mechanism. The U.S. Pat. No. '466 patent varies the mixed ratio of hydrocarbon solvents in an attempt to improve reaction temperature and yield, but there still remains the problem of the base, lithium diisopropylamide being impractical for industrial scale synthesis as it too is expensive, hard to handle, and can be a fire or explosion risk.

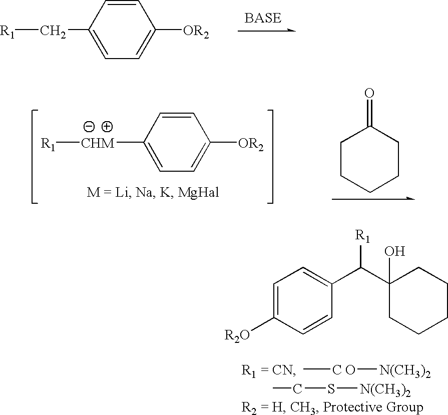

Chinese Patent Publication No. 1225356 (CNO1225356A) discloses the use of bases such as sodium methoxide, sodium ethoxide, sodium hydride and sodium amide in the preparation of cyclohexanol derivatives to enhance the reaction temperature in the range of 0 to 5° C. However, the disclosed bases were used in amounts of at least one equivalent of the reactant, and are also dangerous as they too are prone to combustion or explosion.

The known processes above involve two steps, i.e. reacting phenylacetonitrile with a base to produce an anion and coupling the anion with a ketone compound. In particular, the reaction in the anion-producing step involves some difficulties with regard to determination of the end point of the step and quantitative analysis of the anion produced. Such problems give rise to variations in yield in the coupling step and therefore they are also difficult for industrial application for this reason.

### SUMMARY OF THE INVENTION

Accordingly, the present invention is directed to a process for preparation of cyclohexanol derivatives that substantially overcomes problems and disadvantages of the conventional art.

An object of the present invention is to provide a process for preparation of cyclohexanol derivatives by reaction of phenylacetonitrile with cyclohexanone to enable economical and reasonable mass quantity production.

Another object of the present invention is to provide a process for preparation of cyclohexanol derivatives that is safe and environmentally friendly without the risk of fire or explosion and simpler than conventional syntheses because the reactants are all mixed in one reaction.

One aspect of the present invention is a process for preparation of cyclohexanol derivatives of formula I,

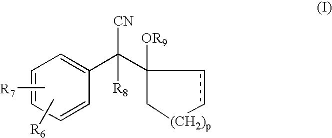

wherein R₆ and R₇ are ortho or para substituents, independently selected from the group consisting of hydrogen, hydroxyl, C₁-C₆ alkyl, C₁-C₆ alkoxy, C₇-C₉ aralkoxy, C₂-C₇ alkanoyloxy, C₁-C₆ alkylmercapto, halo or trifluoromethyl; R₈ is hydrogen or C₁-C₆ alkyl; p is one of the integers 0, 1, 2, 3 or 4; and R₉ is hydrogen or C₁-C₆ alkyl; comprising reacting a compound of formula II with a compound of formula III,

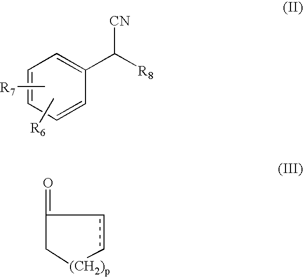

in the presence of non-organometallic base catalysts represented by the formula IV or V, in the presence or absence of a reaction solvent,

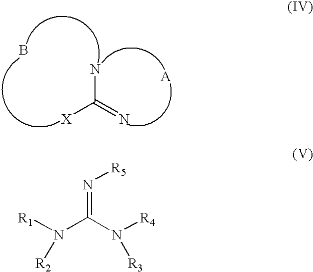

wherein A is —(CH₂)ₙ-where n is an integer from 2 to 4; B is —(CH₂)ₘ— where m is an integer from 2 to 5; X is CH₂, O, NH or NR′ where R′ is a C₁-C₄ alkyl or acyl, or an alkyl supporting polymer; each of and R₁ to R₄ is independently hydrogen, an alkyl, a cycloalkyl or an alkyl or cycloalkyl supporting polymer, and all of R₁ to R₄ are not hydrogen, and R₅ is an alkyl, a cycloalkyl or an alkyl or cyloalkyl supporting polymer, and where R₉ is an alkyl, alkyl group is introduced by alkylation.

The non-organometallic base used in the present invention comprises amidines or guanidines represented by formula IV or V More specifically, examples of non-organometallic bases of the present invention include amidines, e.g. 1,8-diazabicyclo [5,4,0]undec-7-ene (DBU) and 1,5-diazabicyclo[4,3,0]non-5-ene (DBN); cyclic guanidines, e.g. 1,5,7-triazabicyclo[4,4,0]dec-5-ene (TBD) and 7-methyl-1,5,7-triazabicyclo[4,4,0]dec-5-ene (MTBD); alkyl guanidines, e.g. tetra methyl guanidine (TMG), tetra butyl guanidine, penta methyl guanidine, penta butyl guanidine and N′-butyl-N″,N″-dicyclohexylguanidine. The base catalyst of the present invention may be a homogeneous catalyst or may be a catalyst containing an amidine- or guanidine-based organic amine base immobilized on a polymer support (e.g. polystyrene) or an inorganic support (e.g. silica). The non-organometallic base of the present invention is at least one selected from the group consisting of the above-mentioned bases.

The amount of the non-organometallic base used is not specifically limited and may be in the range from about 0.0001 to about 2 equivalents, and more preferably, from about 0.005 to 0.5 equivalents relative to one equivalent of the compound of formula II. The reaction of the present invention can be successfully accomplished with the base catalysts used only in a catalytic amount, which is advantageous.

The present invention may optionally not use an organic solvent comprising hydrocarbons or ethers that are required in conventional synthesis. Whether to use an organic solvent or not is optimally decided by those skilled in the art, but it is generally preferred not to use an organic solvent.

In preparation of the cyclohexanol derivatives such as 1-[cyano(4-methoxyphenyl)methyl]cyclohexanol represented by formula I according to the present invention, the reaction temperature is preferably in the range of about −20 to 80° C., more preferably about 10 to 30° C. The process of the present invention can be conducted even at room temperature, which is advantageous.

The present invention presents a process for preparation of cyclohexanol derivatives by reaction of an appropriately substituted, para-phenylacetonitrile with a cyclohexanone in the presence of a non-organometallic amine base (e.g. DBU, DBN, TBN, MTBD, TMG or N′-butyl-N″,N″-dicyclohexylguanidine) in accordance with reaction mechanism I.

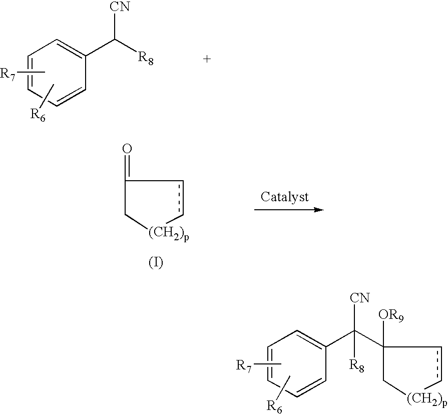

In the above reaction, R₆ to R₉, and p are the same as defined above, and where R₉ is an alkyl, it is introduced by alkylation.

In the preparation of the cyclohexanol derivatives such as 1-[cyano(4-methoxyphenyl)methyl]cyclohexanol represented formula I, a non-organo metallic base such as DBU, DBN, TBD, MTBD, TMG or N′-butyl-N″,N″-dicyclohexylguanidine that is an amine base is used instead of an organometallic base such as n-butyl lithium or lithium diisopropyl amide used in conventional processes to induce a phenylacetonitrile anion. The use of a non-organometallic base, in a relatively small amount that is relatively inexpensive, less susceptible to hydration, operable at room temperature, with no risk of fire or explosion, enables mass quantity production through a safe and relatively simple industrial process. Only catalytic amounts of non-organometallic base are needed in the present invention, which produces highly pure, high yield cyclohexanol derivatives.

The present process is also more simplified and environment-friendly without production of organometallic byproducts as use of organic solvents is avoided.

### DETAILED DESCRIPTION OF THE PREFERRED EMBODIMENTS

The present invention will now be described in detail with reference to the following examples, which are not intended to limit the scope of the present invention.

### EXAMPLE 1

100 g (0.68 mole) of p-methoxyphenylacetonitrile, 100 g (1.02 mole) of cyclohexanone and 32 g (0.21 mole) of 1,8-diazabicyclo [5,4,0] undec-7-ene (DBU) were added to a flask and kept at 15 to 20° C. with stirring for 48 hours. 1N HCl was then added to the resulting solution to regulate the pH to acid level. After one hour of stirring at room temperature, the formed precipitate was separated by filtration and washed with purified water and then with ethyl acetate and n-hexane, to yield 140 g of a white solid as the target compound, 1-[cyano(4-methoxyphenyl)methyl]cyclohexanol (yield 84%, melting point 123.7° C.).

¹H NMR Analysis (DMSO-d6): δ 7.27-6.93 (4H, q, aromatic). 4.85 (1H, s, OH), 4.05 (3H, s, OCH₃), 3.76 (1H, s, CHCN), 1.69-1.08 (10H, m, cyclohexyl)

¹H NMR Analysis (CDCl₃): δ 7.23-6.89 (4H, q, aromatic). 3.82 (3H, s, OCH₃), 3.73 (1H, s, CHCN), 1.72-1.16 (10H, m, cyclohexyl)

¹³C NMR Analysis (DMSO-d6): δ 159.4, 131.3, 125.8, 121.4, 114.1, 72.2, 55.8, 48.8, 36.0, 34.7, 25.9, 22.0, 21.9

Mass Spectral Analysis: Molecular weight 245 [M⁺ by C.I.M.S.]

IR (KBr pallet): 3408 cm⁻¹ (—OH), 2249 cm⁻¹ (—CN)

### EXAMPLE 2

52.7 g (0.36 mole) of p-methoxyphenylacetonitrile, 35.8 g (0.36 mole) of cyclohexanone and 28.6 g (0.19 mole) of 1,8-diazabicyclo[5,4,0]undec-7-ene were added to a flask and kept at 15 to 20° C. with stirring for 90 hours. 1N HCl was then added to the resulting solution to regulate the pH to acid level. After one hour of stirring at room temperature, the formed precipitate was separated by filtration and washed with purified water and then with ethyl acetate and n-hexane to yield 62 g of a white solid as the target compound, 1-[cyano(4-methoxyphenyl)methyl]cyclohexanol (yield 70%).

### EXAMPLE 3

The procedures were performed in the same manner as described in Example 1, except that 0.5 equivalent of 1,8-diazabicyclo[5,4,0]undec-7-ene was used in the 6-day reaction to yield 67 g of a white solid as the target compound, 1-[cyano(4-methoxyphenyl)methyl]cyclohexanol (yield 80%).

### EXAMPLE 4

100 g (0.68 mole) of p-methoxyphenylacetonitrile, 167 g (1.70 mole) of cyclohexanone and 52 g (0.34 mole) of 1,8-diazabicyclo[5,4,0]undec-7-ene were added to a flask and kept at 0° C. with stirring for 60 hours. 1N HCl was then added to the resulting solution to regulate the pH to acid level. After one hour of stirring at room temperature, the precipitate produced was separated by filtration and washed with purified water and then with ethyl acetate and n-hexane to yield 147 g of a white solid as the target compound, 1-[cyano(4-methoxyphenyl)methyl]cyclohexanol (yield 88%).

### EXAMPLE 5

The procedures were performed in the same manner as described in Example 1, except that 0.5 equivalent of 1,8-diazabicyclo[5,4,0]undec-7-ene was used, in the 8-hour reaction to yield 116 g of a white solid as the target compound 1-[cyano(4-methoxyphenyl)methyl]cyclohexanol (yield 70%).

### EXAMPLE 6

25.4 g (0.17 mole) of p-methoxyphenylacetonitrile, 41.8 g (0.42 mole) of cyclohexanone and 13.2 g (0.087 mole) of 1,8-diazabicycle[5,4,0]undec-7-ene were added to a flask and kept at 25° C. with stirring for 24 hours. 1N HCl was then added to the resulting solution to regulate pH to acid level. After adding 50 ml of methyl alcohol and one hour of stirring at room temperature, the precipitate produced was separated by filtration and washed with purified water and then with ethyl acetate and n-hexane to yield 23.7 g of a white solid as the target compound, 1-[cyano(4-methoxyphenyl)methyl]cyclohexanol (yield 56.1%).

### EXAMPLE 7

50.3 g (0.34 mole) of p-methoxyphenylacetonitrile, 34.8 g (0.35 mole) of cyclohexanone and 43.3g (0.35 mole) of 1,5-diazabicyclo[4,3,0]none-5-ene (DBN) were added to a flask and kept at 20 to 25° C. with stirring for 90 hours. To the resulting solution were added 50 ml of methyl alcohol and 200 ml of purified water. After one hour stirring at room temperature, the precipitate produced was separated by filtration and washed with purified water and then with ethyl acetate and n-hexane to yield 116 g of a white solid as the target compound, 1-[cyano(4-methoxyphenyl) methyl]cyclohexanol (yield 70%).

### EXAMPLE 8

20 g (0.14 mole) of p-methoxyphenylacetonitrile, 13.7 g (0.14 mole) of cyclohexanone and 21.2 g (0.14 mole) of 1,S-diazabicyclo[5,4,0]undec-7-ene were added to a flask, diluted with 100 ml of methyl alcohol and kept at 15 to 20° C. with stirring for 20 hours. To the resulting solution were added 20 ml of methyl alcohol and 150 ml of purified water. After one hour of stirring at room temperature, the precipitate produced was separated by filtration and washed with purified water and then ethyl acetate and n-hexane to yield 17.4 g of a white solid as the target compound, 1-[cyano(4-methoxyphenyl)methyl]cyclohexanol (yield 52%).

### EXAMPLE 9

The procedures were performed in the same manner as described in Example 1, except that 0.1 equivalent of 1,8-diazabicyclo[5,4,0]undec-7-ene was used in the 6-day reaction to yield 76.1 g of a white solid as the target compound, 1-[cyano(4-methoxyphenyl)methyl]cyclohexanol (yield 90.5%).

### EXAMPLE 10

25.4 g (0.17 mole) of p-methoxyphenylacetonitrile, 83.6 g (0.85 mole) of cyclohexanone and 26.7 g (0.17 mole) of 1,8-diazabicyclo[5,4,0] undec-7-ene were added to a flask and kept at 20 to 25° C. with stirring for 24 hours. To the resulting solution were added 50 ml of methyl alcohol and 200 ml of purified water. After one hour of stirring at room temperature, the precipitate produced was separated by filtration and washed with purified water and then ethyl acetate and n-hexane to yield 18.0 g of a white solid as the target compound, 1-[cyano(4-methoxyphenyl) methyl]cyclohexanol (yield 42.6%).

### EXAMPLE 11

The procedures were performed in the same manner as described in Example 1, except that the reaction temperature was kept in the range from 35 to 40° C. to yield 30.6 g of a white solid as the target compound, 1-[cyano(4-methoxy-phenyl)methyl]cyclohexanol (yield 36.8%).

### EXAMPLE 12

100 g (0.68 mole) of p-methoxyphenylacetonitrile, 100 g (1.02 mole) of cyclohexanone and 0.47 g (0.0034 mole) of 1,5,7-triazabicyclo[4,4,0] dec-5-ene (TBD) were added to a flask and kept at 20 to 25° C. with stirring for 10 to 12 hours. 1N HCl was then added to the resulting solution to regulate the pH to acid level. After one hour of stirring at room temperature, the precipitate produced was separated by filtration and washed with purified water and then ethyl acetate and n-hexane to yield 128 g of a white target compound, 1-[cyano(4-methoxyphenyl)methyl]cyclohexanol (yield 77%).

### EXAMPLE 13

The procedures were performed in the same manner as described in Example 1, except that 0.03 equivalent of 7- methyl-1,5,7-triazabicyclo [4,4,0] dec-5-ene (MTBD) was used in the 20 to 22-hour reaction to yield 128 g of a white solid as the target compound, 1-[cyano(4-methoxyphenyl)methyl]cyclohexanol(yield 77%).

### EXAMPLE 14

50 g (0.34 mole) of p-methoxyphenylacetonitrile, 50 g (0.51 mole) of cyclohexanone and 0.24 g (0.0017 mole) of 1,5,7-triazabicyclo[4,4,0] dec-5-ene (TBD) were added to a flask and kept at 20 to 25° C. with stirring for 19 hours. The reaction mixture was dissolved in 500 ml of ethyl acetate and, after addition of 200 ml of purified water, was neutralized with 6N HCl. Following phase separation at 30 to 35° C., the organic solvent was removed under vacuum and 500 ml of ethyl acetate and 200 ml of purified water were added to the filtrate. After one hour of stirring at room temperature, the precipitate produced was separated by filtration and washed with purified water and then with ethyl acetate and n-hexane to yield 74 g of a white solid as the target compound, 1-[cyano(4-methoxyphenyl)methyl]cyclohexanol (yield 89%).

### EXAMPLE 15

25 g (0.17 mole) p-methoxyphenylacetonitrile, 25 g (0.25 mole) cyclo-hexanone and 2.5 g (0.0090 mole) N′-butyl-N″,N″-dicyclohexylguanidine were added to a flask and kept at 20 to 25° C. with stirring for 24 hours. 1N HCl was then added to the resulting solution to regulate the pH to acid levels. After one hour of stirring at room temperature, the precipitate produced was separated by filtration and washed with purified water and then with ethyl acetate and n-hexane to yield 30 g of a white solid as the target compound, 1-[cyano(4-methoxyphenyl)methyl]cyclohexanol (yield 72%).

### COMPARATIVE EXAMPLE 1

50 g (0.34 mole) of p-methoxyphenylacetonitrile was diluted with 250 ml of dry tetrahydrofuran (THF) and cooled to −70° C. under a nitrogen atmosphere. 210 ml (0.34 mole) of n-butyl lithium (n-BuLi) was dropped into the resulting solution while maintaining the temperature of the solution below −50° C. The solution was then stirred for 30 minutes and, after addition of 50 g (0.51 mole) of cyclohexanone, was stirred for 45 minutes more, while the temperature of the solution was kept at less than −50° C. Thereafter, the temperature of the reaction solution was raised to 0° C., and a saturated ammonium chloride solution was added to cause phase separation. The aqueous layer was extracted with diethyl ether and combined with the organic layer. The organic solvent was then removed under reduced pressure to yield 25.2 g of the target compound, 1-[cyano(4-methoxyphenyl)methyl]cyclohexanol (yield 34.2%).

Melting point: 123 to 126° C.

Mass Spectral Analysis: Molecular weight 245 [M⁺ by C.I.M.S.]

¹H NMR Analysis (DMSO-d6): δ 7.32, 6.95 (4H, q, p-substituted aromatic), 3.8 (3H, S, O—CH₃), 3.76 (1H, s, CH—CN), 1.56 (10H, m, aliphatic cyclohexyl)

### COMPARATIVE EXAMPLE 2

While maintaining internal temperature below 10° C., 76.5 g of p-methoxyphenylacetonitrile diluted with 75 ml of toluene was slowly added to lithium diisopropylamide solution made by adding 73 ml of diisopropylamine to 325 ml of 6M BuLi and 300 ml of toluene under a nitrogen atmosphere. After 30 minutes of stirring, 46.0 g of cyclohexanone diluted with 50 ml of toluene was slowly added with the internal temperature kept below 10° C., and stirred for more than about 30 minutes. The resulting solution was then added to 100 ml of 12N HCl aqueous solution and 1 L of cold purified water. After filtration, the filtrate was diluted with dichloromethane and washed with purified water. With the dichloromethane replaced with diisopropyl ether, the solvent was removed under reduced pressure and the filtrate was cooled down and filtered to yield 91.0 g of a white solid as the target compound, 1-[cyano(4-methoxyphenyl)methyl]methyl]cyclohexanol (yield 79%).

### Reference Example

12 g (0.05 mole) of 1-[cyano(4-methoxyphenyl)methyl]cyclohexanol prepared in Example 1 was dissolved in 250 ml of a mixture of ammonia and ethanol, with the mixing ratio of 2:8 (v/v), and 2.8 g of 5% rhodium on alumina was added to cause a hydrogenation reaction. The catalyst was filtered out and washed with ethanol and the filtrate was concentrated under reduced pressure to provide a compound in the form of oil, which was then diluted with 100 ml of toluene and acidified to pH 2. After filtration, 9 g of a white solid was obtained as the target compound, 1-[2;-amino-1-(4-methoxyphenyl)ethyl]cyclohexanol (yield 57%).

Melting point: 168 to 172° C.

Mass Spectral Analysis: Molecular weight 250 [M⁺ by C.I.M.S.]

¹H NMR Analysis (DMSO-d6): δ 7.85 (3H, s, NH³⁺), 3.75(3H, s, O—CH₃), 3.20 (3H, m, CHCH₂), 1.35 (10H, m, aliphatic cyclohexyl)

As described above, the present invention provides a safe and relatively simple process for industrial scale mass quantity production of cyclohexanol derivatives such as 1-[cyano4-methoxyphenyl]methyl]cyclohexanol represented by formula I. The present invention uses a relatively inexpensive, non-metallic base in small amounts, which is environment-friendly and avoids organic solvents, to produce highly pure 1-[cyano4-methoxyphenyl]methyl]cyclohexanol in high yield.

What is claimed is:

1. A process for preparation of cyclohexanol derivatives of formula I 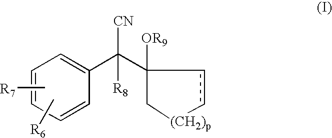
wherein R₆ and R₇ are ortho or para substituents, independently selected from the group consisting of hydrogen, hydroxyl, C₁-C₆ alkyl, C₁-C₆ alkoxy, C₇-C₉ aralkoxy, C₂-C₇ alkanoyloxy, C₁-C₆ alkylmercapto, halo or trifluoromethyl; Rs is hydrogen or C₁-C₆ allyl; p is one of the integers 0, 1, 2, 3 or 4; and R₉ is hydrogen or C₁-C₆ alkyl; comprising reacting a compound of formula II with a compound of formula III, 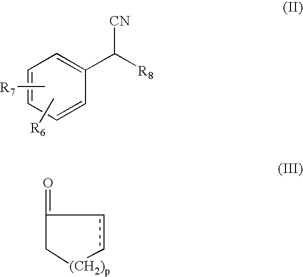
in the presence of a non-organometallic base catalyst represented by formula IV or V, in the presence or absence of a reaction solvent: 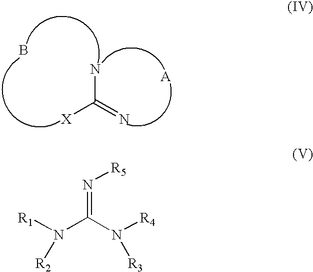
wherein A is —(CH₂)ₙ— where n is an integer from 2 to 4; B is —(CH₂)ₘ— where m is an integer from 2 to 5; X is CH₂, O, NH or NR′, where R′ is a C₁-C₄ alkyl or acyl, or an alkyl supporting polymer; and each of R₁ to R₄ is independently hydrogen, an alkyl, a cycloalkyl or an alkyl or cycloalkyl supporting polymer and all of R₁ to R₄ are not hydrogen, and R₅ is an alkyl, a cycloalkyl or an alkyl or cycloalkyl supporting polymer, and where R₉ is an alkyl, alkyl group is introduced by alkylation.

2. The process of claim 1, wherein the compound of formula II is p-methoxy-phenylacetonitrile.

3. The process of claim 1, wherein the compound of formula III is cyclohexanone.

4. The process according to any one of claims 1 to 3, wherein the non-organometallic base catalyst is a mixture of catalysts selected from one or more amidines or guanidines of formula (IV) or (V).

5. The process according to any one of claims 1 to 4, wherein the base catalyst is either homogeneous or immobilized on a polymer support.

6. The process according to any one of claims 1 to 5, wherein the non-organo-metallic base is selected from the group consisting of 1,8-diazabicyclo[5,4,0]undec-7-ene (DBU), 1,5-diazabicyclo[4,3,0]non-5-ene (DBN), 1,5,7-triazabicyclo[4,4,0]dec-5-ene (TBD), 7-methyl-1,5,7-triazabicyclo[4,4,0]dec-5-ene (MTBD), tetra methyl guanidine (TMG) and N′-butyl-N″,N″-dicyclohexylguanidine.

7. The process according to any one of claims 1 to 6, wherein the amount of the non-organometallic base used is in the range from about 0.005 to about 0.5 equivalents relative to one equivalent of the compound of formula II.

8. The process according to any one of claims 1 to 7, wherein no solvent is used.

9. The process according to any one of claims 1 to 8, wherein the reaction temperature is in the range of about −20 to 80° C.

10. The process of claim 9, wherein the reaction temperature is in the range of about 10 to 30° C.

11. The process according to any one of claims 1 to 10, wherein the compounds of formulas II and III and the base catalysts are used in equivalent ratios of 1:1˜1.5: 0.005˜0.5.
